(19) United States
(12) Patent Application Publication
(10) Pub. No.: US 2020/0200739 A1
(43) Pub. Date: Jun. 25, 2020

(54) URINE SAMPLING KIT AND METHOD

(71) Applicant: PALMA GROUP SA, Fribourg (CH)

(72) Inventor: Pavlo PORVAN, Fribourg (CH)

(21) Appl. No.: 16/614,775

(22) PCT Filed: Nov. 1, 2017
(86) PCT No.: PCT/IB2017/056789

§ 371 Date: Nov. 18, 2019

(30) Foreign Application Priority Data
May 24, 2017 (CH) 685/17

(51) Int. Cl. G01N 33/52; G01N 21/78; A61B 10/00; A61F 13/505

(52) U.S. Cl. CPC G01N 33/528; G01N 21/78; A61F 2013/8473; A61F 13/505; A61B 2010/0006; A61B 10/007

(57) ABSTRACT

The present invention relates to a method for identifying deviations from a standard composition of urine and from standard physicochemical parameters, which method comprises separating a urine collection element from a diagnostic layer, wetting a urine indicator strip via the urine collection element, and reading the result of the analysis by comparison against a colorimetric scale. The invention likewise relates to an analysis kit for identifying any deviations from a standard.

### TECHNICAL FIELD

The invention relates to a method for the qualitative identification of compounds in human urine. In particular, it makes it possible to identify the presence of compounds that are normally absent, such as glucose, ketones, albumin, leucocytes, monocytes or erythrocytes. The present invention further allows the identification of deviations from the norm of certain urine indices such as, for example, pH.

The present invention also covers the kit of elements necessary for the implementation of the method.

The invention can be used for research purposes in the field of medicine, as well as for diagnosing the health status of human beings. The diagnosis is then based on the analysis of the deviation from the norm of urine indices, or the identification of one or more compounds.

The invention can be used both in medical institutions and by non-professionals in domestic conditions.

### PRIOR ART

Detecting an early-stage pathology significantly facilitates its treatment in adults and children for whom diseases can develop rapidly. In particular, the first year of a child's life can be crucial in the evolution of his health. The steady rise in the level of childhood illnesses and morbidity is a concern for many governments and has led Canada to initiate a government program to test the urine and blood of newborns.

Urinalysis can diagnose various disorders or identify different pathologies. This makes it possible to quickly initiate prophylaxis and/or the appropriate treatment. Therapeutic indications include metabolic disturbances, obesity, diabetes mellitus, rotavirus infection, genetic diseases, disturbances of the thyroid function. For children still wearing diapers, especially for children aged 0 to 18 months, it may be difficult to collect urine for analysis. Regardless of age, the problem also arises for people whose physical possibilities are limited. In particular, it may be difficult to collect a urine sample of elderly, disabled, bedridden, or mobility-impaired persons. In fact, known methods require collecting the urine sample in a container before carrying out the analysis. In practice, the sample is analyzed by immersion of urine indicator strips in the urine collected. The existing urine indicator strips are in the form of plastic strips on which reagents are placed. They are generally packaged in tubes, each comprising 50 to 100 strips. Urine strip tubes have a moisture absorber. Such an arrangement, while it is suitable for frequent tests performed in laboratories or for chronic patients requiring regular monitoring, proves impractical for home use. Home-based operations are often limited to collecting the urine sample in a container, which must then be taken to an analytical laboratory.

The volume of urine to be collected must be sufficient to allow the immersion of all the reagents of the urine test strip. The evaluation of the results of the urine analysis is based on the change of the color of one or more indicator reagents under the action of the urine compounds. The colors of the reagents are then compared to a color scale. The reagents placed on the urine indicator strip are each specific for the urine compound whose presence is sought. The usual method of immersion/absorption requires manipulation of the patient for the collection of the sample to be analyzed. This manipulation can be difficult and sometimes restrictive during repeated analyzes for daily monitoring. The usual method of immersion/absorption also requires that the urine strip be removed from the sample sufficiently rapidly to prevent the reagents from degrading or dispersing in the sample. In addition, the removal of the urine strip from the container containing the sample inevitably leads to a flow of urine along the urine strip, which can lead to the mixing of the reagents disposed on the urine indicator strip. It is therefore necessary to quickly dispose the strip horizontally to prevent the flow of urine on the reagents. Placing the strip soaked with the patient's urine on a horizontal support can easily violate hygiene rules.

The usual method of immersion/absorption is illustrated in FIG. 1.

### PRESENTATION OF THE INVENTION

The present invention overcomes the aforementioned drawbacks.

In particular, the method implemented in the present invention makes it possible to perform urine analysis simply by accumulating urine in an accumulation element, which is then used to moisten a urine indicator strip.

The accumulation element is preferably removably disposed on a diaper, so that it is not necessary to manipulate the patient in order to collect a urine sample. The method of wetting/accumulating includes, in particular, a step of detaching the accumulation element, a step of moistening a urine indicator strip by the accumulation element detached from the diaper, and a step of observing the colors of the urine indicator strip, and comparison with a color scale.

The urine accumulation element may comprise a removable attachment means and an absorption device. The detachment of the urine accumulation element then includes the detachment of the removable attachment means, which allows the absorption device soaked in urine to be taken to be analyzed.

The removable attachment means is disposed in a central and outer region of the diaper, thereby providing access to a urine sample without having to remove the diaper.

The urine indicator strip is preferably associated with, or disposed on, an analysis device comprising a test area, an area having one or more color scales, an area for disposition of the urine accumulation element, an area for disposition of a moisture absorber, and a raised extrusion area.

The moistening of the urine indicator strip by the urine accumulation element is maintained for a period of 10 to 120 seconds.

The reading of the results can optionally be performed after a delay of between 0 and about 120 seconds.

The reading of the result may include the comparison of the color of the indicator reagents with the corresponding color scale(s). Alternatively or in addition, the reading of the result may include the recognition of the color of the indicator reagents by an electronic device equipped with an interface.

The present invention further comprises a diagnostic diaper comprising at least one urine accumulation element, disposed outside the diaper and in a central position, and which is removably attached to the diaper by a removable attachment means.

The present invention further comprises an analysis device comprising at least one test area, an area having one or more color scales, and an area for disposition of a urine accumulation element. An additional area for the provision of a moisture absorber and/or a raised extrusion area may be provided on the analysis device. The analysis device preferably comprises an area dedicated to the disposition of a moisture absorber.

The present invention further comprises an analysis kit comprising at least the diaper and the analysis device as described above. The analysis kit advantageously contains one or more urine indicator strips, which may be free or attached to the analysis device. The analysis device is preferably packaged in an airtight package. It may be individually packaged. The kit according to the invention may further comprise an explanatory note or a reference to a downloadable program on an electronic device, or an explanatory note and a reference to a downloadable program on an electronic device.

### BRIEF DESCRIPTION OF THE FIGURES

FIG. 1: Schematic representation of the traditional urine sampling method and its analysis in a child aged 0 to 18 months.

FIG. 2a, 2b, 2c, 2d: Representations of the diaper with the urine accumulation element, during urine collection steps, according to the present invention.

FIG. 3: Schematic representation of the analysis device according to the present invention.

FIG. 4: Schematic representation of the implementation of the analysis method according to the present invention.

FIG. 5: Schematic representation of the analysis device and its sealed package after opening.

FIG. 6: Schematic representation of the interface of an electronic device for the conduct of the analysis.

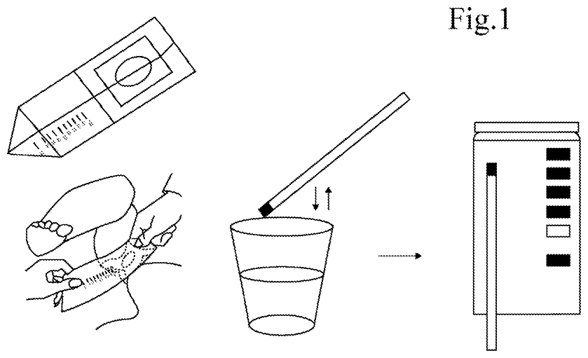

### DETAILED DESCRIPTION OF THE INVENTION

The present method of urine analysis makes it possible to identify possible deviations from the norm of one or more of its compounds or parameters, thanks to the principle of HUMECTATION/ACCUMULATION. The analysis is preferably qualitative. The deviation from the norm of one or more of the urine compounds refers to the presence of compounds normally absent from the urine, or their presence in a different amount from a predetermined threshold. An amount greater than the norm of one or more urine compounds is preferentially detected, but an insufficient amount with respect to the norm can also be detected.

Urine compounds refer to all the chemical and biological elements present in the urine at the time of testing, including unmetabolized excess products and metabolic breakdown products.

Throughout this description and unless otherwise stated, the norm is understood as being a reference value corresponding to the usual value observed in the population, such a reference value reflecting a suitable state of health of the person at the origin of the sample. The deviation from the norm therefore means a difference with respect to this norm for the compound or parameter being measured, and may be indicative of a malfunction or pathology. The compounds of urine include sugars, and, in particular, glucose and its derivatives, albumin, peptides and proteins as well as amino acids, nitrogen derivatives such as urea, ketones, cells and cells such as leukocytes and red blood cells.

Parameters other than urine compounds can be determined in this method. In particular, the pH can be evaluated to determine the possible deviation from the norm.

Determining a deviation or absence of deviation from the norm involves the reaction of a colored indicator, preferably a specific chemical, of the urine compound to be analyzed. It is understood that any urine compound or physicochemical parameter that can be detected by reaction with a colored indicator can be analyzed according to the present invention.

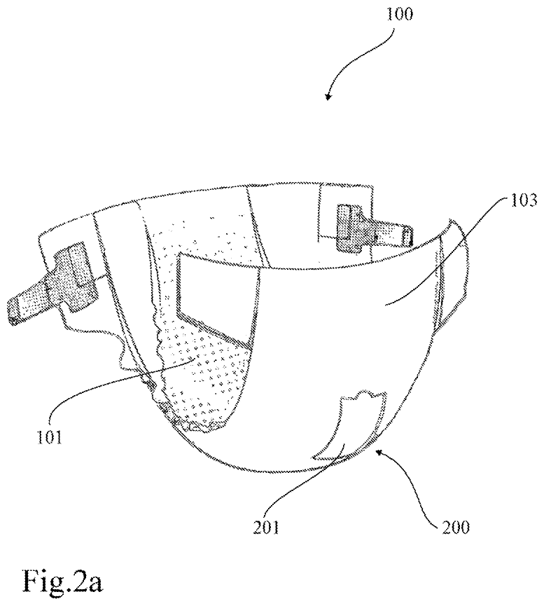

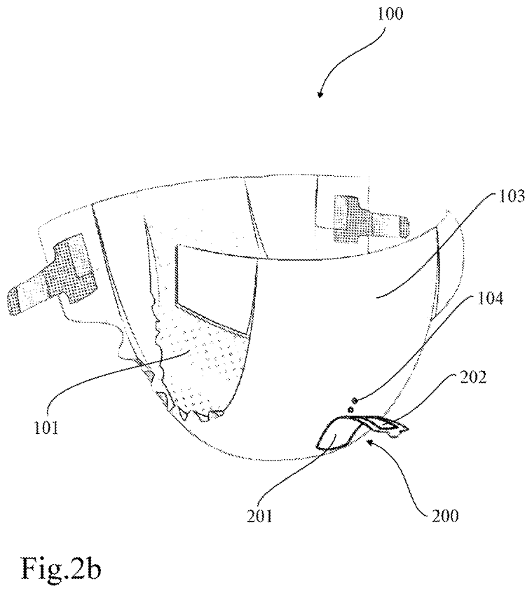

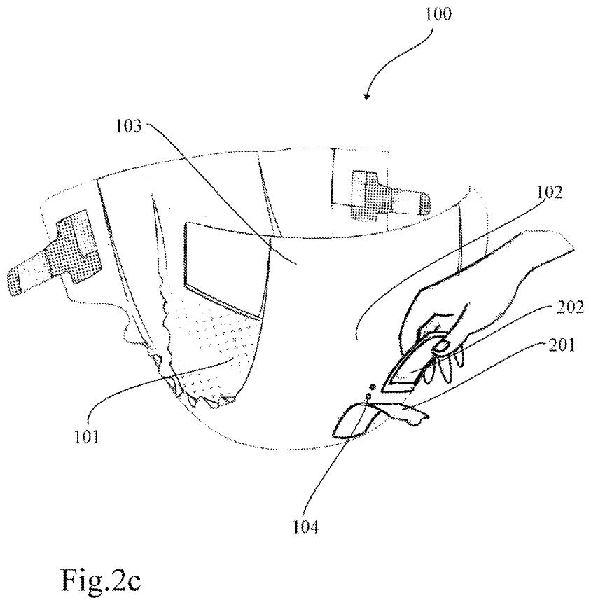

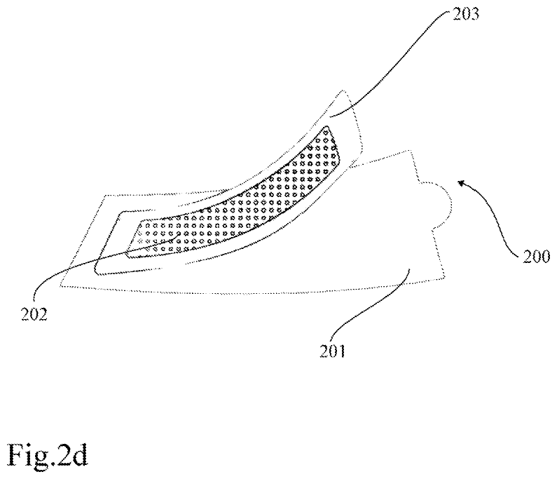

The present method involves the use of a diaper 100, comprising a urine accumulator 200. The diaper 100 is thus adapted to the collection of biological samples, such as urine. Such a diaper may be that described in detail in the patent application CH 00429/17 filed on 30 Mar. 2017 and shown in FIGS. 2a, 2b and 2c. The urine accumulator 200 collects sufficient urine to react the indicator reagents 307 by moistening a strip on which one or more indicator reagents 307 are disposed. A quantity of the order of 2 to 3 ml of urine can thus be collected by means of the urine accumulator 200. An amount of the order of 3 ml of urine can thus be collected in certain diaper models through the urine accumulator 200.

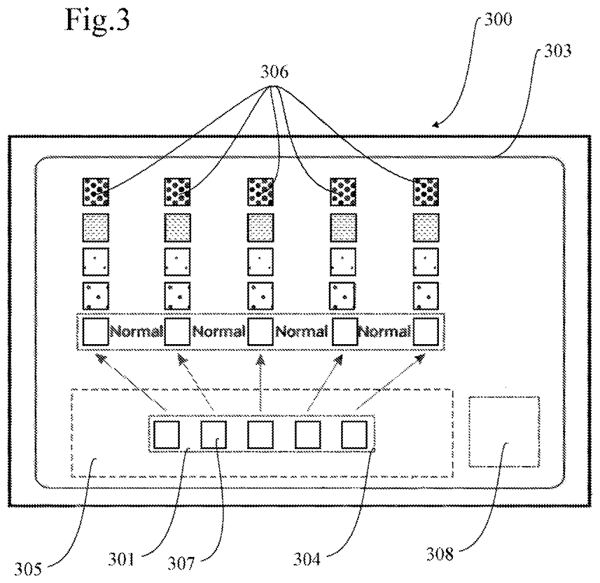

The present method also requires the use of an analysis device 300 in which a urine indicator strip 301 is disposed, an example of which is shown in FIG. 3. The analysis device 300 can for example be in the form of a plate or board.

The analysis device 300 is preferably packaged in a hermetic package 302. The hermetic package 302 advantageously comprises a single analysis device 300, thus making the packaging individual. However, it is not excluded that several analysis devices 300 may be included in the hermetic package 302 depending on the intended uses. The analysis device 300 may be made of laminated paper and have a raised extrusion 303 at the edges to prevent the urine overflowing. This analysis device 300 has in particular the following areas:
Test area 304, in which is disposed a urine strip 301 comprising indicator reagents 307 (test area)
Area 305 for disposition of the urine accumulator 200
Colorimetric area 306
Area 308 for disposition of the moisture absorber 3
Relief extrusion area 303

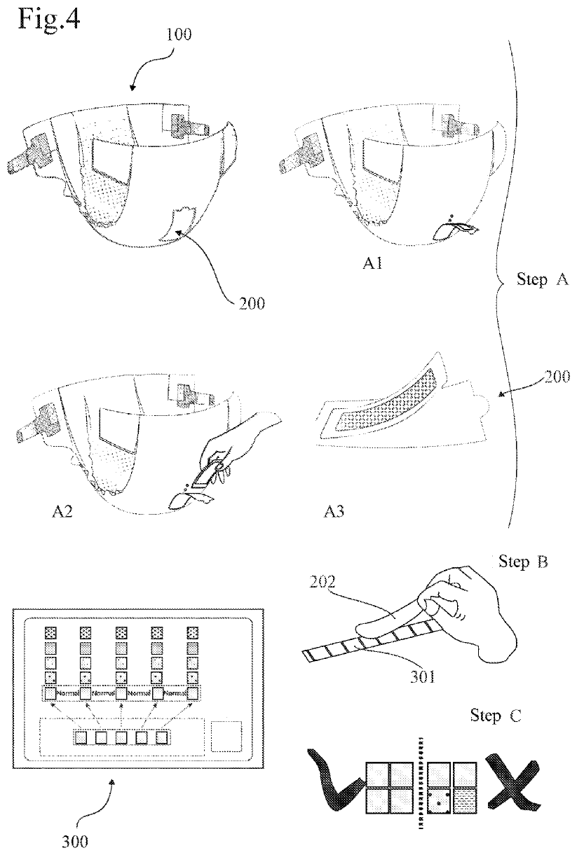

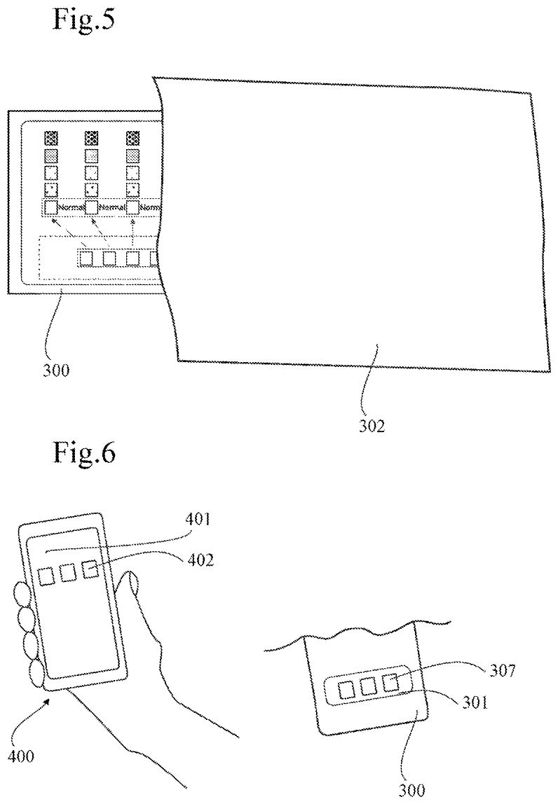

The main steps of the analysis method are illustrated in FIG. 4. The analysis method comprises, in particular, a step A for separating the accumulation element 200 from the diagnostic diaper 100, a step B of moistening a urine strip 301 by the accumulation element, and a step C of reading the result of the analysis on the urine strip 301.

Step A may comprise one or more of the intermediate steps A1 for separating a removable fastening element 201, A2 for removing an absorption device 202 integral with the removable fastening element 201, and A3 for separating the absorption device 202 from a fastening interface 203. In the intermediate step A1, the removable fastening element 201 can be removed completely from the diaper 100, or only partially. It should be noted that in the case where the absorption device 202 can be easily detached from the removable fastening means 201, the latter can be removed from the diaper 100 in a partial manner, and thus allows the removal of the absorption device 202 during the intermediate step A2. Alternatively, the removable fastening element 201 may be totally removed from the diaper 100 with the absorption device 202. Under these conditions, the intermediate steps A2 and A3 may be optional, insofar as the absorption device 202 may be directly used for the wetting of the indicator reagents 307, although still being connected to the removable fastening element 201. In the case where the absorption device 202 comprises a fastening interface 203 which keeps it associated with the removable fastening element 201, an intermediate step A3 can be provided for separating the fastening interface 203. The step A described above, as well as the intermediate steps A1, A2 and A3 can take place while the diaper 100 is still in place on the person, which enables manipulation to be limited.

The step B of wetting the strip 301 includes intermediate steps B1, application of the absorption device 202 on the strip 301 comprising one or more indicator reagents 307, and B2 removal of the absorption device 202. As indicated above, the absorption device 202 may be applied alone or in combination with one or more of the other constituents of the urine accumulator 200, such as the removable fastening element 201 and the attachment interface 203. The wetting of the step is preferably performed when the urine strip 301 is disposed in the test area 304 of the analysis device 300. A preliminary intermediate step B3 may be provided during which the user places the urine test strip 301 in the test area 304. This intermediate step B3 is made optional when the strip 301 is fixed on the analysis device 300 before it is packaged in the package 302. A preliminary step B4, including the opening of the package 302, is also provided prior to the implementation of the analysis method according to the present invention. The intermediate step B2 of removal of the absorption device 202 of the urine strip 301 is carried out after a predetermined duration of application, sufficient to react all the indicator reagents 307 of the urine strip 301. The intermediate step B2 of removal of the absorption device 202 therefore occurs after a wetting period of between 10 and 120 seconds, preferably between 60 and 120 seconds, and more particularly between 30 and 60 seconds. The wetting time is, in particular, adapted to the reaction rate of the indicator reagents 307. The wetting is interrupted after a period sufficiently long to react the indicator reagents 307 and sufficiently short to prevent degradation by the urine.

Step C of reading of the result of the analysis may include an intermediate timing step C1, during which the color of the indicator reagents 307 continues to change after removal of the absorption device 202 of the urine strip 301. This period can vary from 0 to 120 seconds depending on the reaction rate of the indicator reagents 307. The intermediate step C1 is preferably between 30 and 60 seconds. The intermediate timing step C1 may alternatively be left to the user who can decide that the indicator reagents 307 can be read when their color no longer varies. Preferably, the result can be obtained after about 1 minute after the beginning of the wetting. Step C may further comprise an intermediate step C2 for comparing the color of the reagent or reagents 307 disposed on the urine strip 301 with one or more colorimetric scales 306. This intermediate comparison step C2 makes it possible to identify a possible difference compared to the norm, materialized by a reference color. Step C of reading the result may additionally or alternatively comprise an intermediate step C3 of electronic analysis, involving the use of a suitable electronic device and a suitable analysis program. This intermediate step C3 comprises a color recognition of the indicator reagents 307 by an electronic means, such as a camera or a photographic module, as well as the interpretation of the recognized color. The electronic device 400 can then inform the user of a possible deviation from the norm of one or more of the compounds and parameters analyzed, or of non-deviation from the norm. The information can be delivered to the user of the electronic device 400 by means of a written message, visible on the interface 401 of the application, or delivered by email, or by an audio message, or by a combination of written and audio messages. Simplified information, consisting of the identification of deviation or non-deviation from the norm of one or more of the compounds and parameters of the urine, can be materialized by an “OK” message in case of non-deviation, or “Non-Compliant” in case of deviation. Any equivalent message or predetermined color code, such as green in case of non-deviation and red in case of deviation, may be used. More complete information identifying the compound(s) and urine parameter, and their status “OK” or “Non-Compliant”may alternatively be issued to the user of the electronic device or to a person authorized by the user of the electronic device.

Although one or the other intermediate steps C2 and C3 are sufficient to read the result of the analysis, they can be implemented concomitantly, thus allowing a double reading.

In case of deviation from the “Norm” portion of the color scales 306, the analysis may be repeated after two hours and at least one hour before or after a meal. Indicator reagents 307 are those commonly used for the analysis of urine compounds. An indicator reagent 307 of the urine strip 301 may contain, for example, sodium nitroprusside, so as to detect ketones possibly contained in the urine sample. Other usual indicator reagents can be used.

The moisture absorber 3 is preferably attached to the device 300, which avoids its direct influence on the indicator reagents 307 in the individual package 302.

The analysis device 300 is provided for one or more components or parameters.

Preferably, the analysis device 300 is provided for at least 3 components and/or parameters. For example, an analysis device 300 may be designed to determine the presence of glucose and ketones in the urine, and to measure the pH. In addition or instead, other components such as leukocytes, erythrocytes and albumin can be studied. All combinations are also possible.

The present invention provides an analysis kit K comprising the elements necessary for the implementation of the method described above. The analysis kit K comprises, in particular, a diagnostic diaper 100 and an analysis device 300.

The diagnostic diaper 100 comprises an absorbent material over most of its absorption surface 101. A central area 102 of its absorption surface 101 is preferably free of absorbent material, or provided with a smaller amount of absorbent material. The diaper 100 is provided with an impermeable outer envelope 103 pierced with one or more orifices 104 opposite the central area 102, and the absorbing properties of which may be reduced relative to the remainder of the absorption area 101. The diagnostic diaper 100 further comprises an accumulation element 200 removably disposed on the orifices 104 of the impermeable envelope 103. The accumulation element 200 comprises a removable fastening element 201, which enables it to be kept in place on the diaper 100, and an absorption device 202 which absorbs the urine not retained by the absorption area 101 of the diagnostic diaper 100. The absorption device 202 is preferably integral with the removable fastening element 201. Alternatively, the absorption device 202 can be separated from the removable fastening element 201.

The removable fastening element 201 can take, for example, the form of a tongue provided with an adhesive substance on its face affixed to the diagnostic diaper 100. The tongue, wider than the orifice or the holes 104 of the impermeable envelope 103, can then be glued to the diagnostic diaper 100 and maintain the absorption device 202 in front of the orifice 104. The absorption device 202 may in turn comprise one or more diapers of an absorbent material in woven or non-woven form. An absorbent material that is neutral with respect to the components of the urine to be studied, is preferably used, so as not to degrade the sample to be analyzed. A plant or synthetic fiber, or a mixture can be used. The absorption device 202 may further comprise one or more hygroscopic products, provided that they do not interact with the desired urine components, or retain them to the point of preventing their reaction with the indicator reagents 307. In the case where the absorption device 202 can be separated from the removable fastening element 201, it can comprise a fastening interface 203 which makes it possible to retain it on the internal face of the removable fastening element 201.

The analysis device 300 is preferably pre-packaged in an individual hermetically-sealed package 302. The package in question may be made, in particular, of a plastic material that is impermeable to air and moisture, and preferably opaque to light, in order to preserve the indicator reagents 307. The package 302 may further comprise one or more pre-cut notches, to facilitate opening. The package 302 may also contain an explanatory note 310. Alternatively, the explanatory note 310 may be affixed outside the package 302, or included separately in the analysis kit K.

The package 302 contains the analysis device 300, at least one urine strip 301 provided with at least one indicator reagent 307, and a moisture absorber 3.

The analysis device 300 may be made of a cellulose material, such as cardboard or paper, or plastic. It can be rigid or flexible. It preferably comprises an analysis area delimited by a relief 303 intended to contain the possible excess of urine in the analysis area. The analysis area comprises at least one test area 304, where a urine test strip 301 can be arranged. The test area coincides at least in part with an area 305 of disposition of the urine accumulator 200, such that the urine strip 301 is moistened upon application of the urine accumulator 200. The analysis area of the device may further contain one or more colorimetric scales 306. Alternatively, the color scale(s) 306 are placed outside the analysis area to avoid being soiled by any traces of urine. The analysis device 300 has as many colorimetric scales 306 as the urine strip 301 contains indicator reagents 307.

To facilitate the reading of the diagnosis, the colorimetric scales 306 are arranged opposite the corresponding indicator reagents 307. In order to avoid reading errors, the urine strip 301 is preferably fixed in advance on the analysis device 300, so that the indicator reagents 307 are associated with their corresponding color scale. The color scales 306 may be accompanied by a caption indicating the product or the parameter to which it is dedicated. The color scale(s) 306 preferably include a reference color indicating the norm of the product or corresponding parameter. The reference color then corresponds to the color of the indicator reagent 307 of the urine strip 301 after the implementation of the present method, when the compound or the parameter measured does not show any deviation from the norm. The scale or color scales 306 comprise at least one additional color, different from the reference color, and indicative of a deviation from the norm. There may be other indicator colors for greater deviations from the norm.

The colors indicating the greatest deviations from the norm are preferably arranged on the color scales 306 at a greater distance from the reference color than the indicator colors with smaller deviations from the norm.

The color of the reagents 307 of the urine strip 301 obtained after its wetting can then be compared with the colors of the corresponding color scale, in order to identify a possible deviation from the norm.

In case the analysis device 300 contains several colorimetric scales 306, their colors are specific of the corresponding indicator reagents 307. They can therefore vary from one color scale 306 to another.

The analysis device 300 may further comprise a positioning area 308 of a moisture absorber 3. The moisture absorber 3 is preferably fixed to the analysis device 300 at its positioning area 308, at the time of its packaging, in order to keep the atmosphere of the individual package sufficiently dry and thus limit or prevent the degradation of the indicator reagents 307.

The moisture absorber may contain hygroscopic elements of natural and/or synthetic origin. These elements can be packaged in a container permeable to air or fixed on the moisture absorber 3. The attachment of the moisture absorber 3 on the analysis device 300 avoids contact with the indicator reagents 307.

The analysis device 300 may comprise an instruction area in addition to the analysis area. The instruction area reminds the user, for example, of the steps to be taken to perform the analysis of the urine sample, or refer to the instructions provided in a separate user manual. The information area can additionally or alternatively include a digital recognition sign such as an OR code, which allows the user to access digital files. Such digital files may contain, for example, one or more videos or instructions to be followed in case of an abnormal situation. The information area may furthermore alternatively include a free space allowing the user to make annotations, such as the date and time of the test, or the surname and first name of the person at the origin of the sample.

The instructions for use included in the analysis kit K contain the information necessary for the implementation of this diagnostic method. It may also include warnings and/or safety or hygiene instructions. The instructions for use may also contain references to an electronic program accessible to the user. Such a program can be downloaded to an electronic device 400, such as an electronic tablet, a mobile phone or any other suitable electronic device. Such a program may be, in particular, a mobile application making it possible to facilitate the diagnosis via an appropriate interface 401. The program enabling the diagnosis in an electronic manner may, in particular, comprise one or more detection areas 402 materialized on the screen of the electronic device 400. and corresponding to the indicator reagents 307 of the urine strip 301. The camera module of the electronic device 400, or any other comparable device, makes it possible to make these detection areas 402 coincide with the indicator reagents 307 of the urine strip 301 and to capture an image of them. The color of the indicator reagents 307 is thus detected and compared with reference values previously recorded in the electronic program. The result of the test can then be rendered in the form of a written and/or audio message via the electronic device 400. The electronic program can furthermore include a function of monitoring the results to establish a history and to detect any variations in the analyses.

The analysis results may or may not be recorded on a remote server of the electronic device 400 used to perform the test. Warning messages may also be provided to induce the user to perform the test or to repeat it. The electronic program may include one or more levels of delivery. A first level of service may be defined, for example, for occasional users and a second level of service, offering more functionalities, may be intended for health professionals.

In the case where the analysis kit K includes a reference to an electronic program accessible to the user, the color scales 306 of the analysis device 300 may be optional. In the case where they are present, the user can compare the result obtained via the electronic device 400 with the result obtained by the observation of the color scale 306, thus allowing a double control.

The present invention also covers the separate elements of the analysis kit K described above. In this case, the present invention comprises a diagnostic diaper 100 provided with a removable urine accumulator 200, as well as the urine strip 301. It should be noted that the urine strip 301 used in the present method may be of reduced length and include indicator reagents 307 along its entire length. It is, in fact, not necessary to dedicate an end to its manipulation, which is devoid of indicator reagents.

The method according to the invention makes it possible, by means of screening the urine, to identify, at their initial stages, any disturbances in the state of health of a person. A specialist can then be contacted for consultation and treatment. The method also allows simple and regular monitoring of dysfunctions or pathologies already identified. It can be used, in particular, to determine possible deviations from the evolution of the pathology, or to identify the best times to take medication. This method can also be used routinely during treatments or clinical tests in order to monitor their effects on the body.

The diagnostic diaper 100 is particularly advantageous for collecting urine in the most comfortable conditions for the person whose urine is analyzed and for the person performing the test.

This method does not require any special knowledge and can be implemented at home at any time by the patient's relatives. Parents can easily monitor the health status of their child.

The color scales 306 used may be identical to those available on the market, which does not require special training of health personnel.

### Validity of the Method

In one study, the present invention is reproduced under the conditions of a clinical laboratory. The purpose of this study is to determine the validity of the urine results obtained by the wetting method of indicator reagents by comparing these results with those obtained using the traditional urine dip tube, i.e. a container containing the urine. In this study, urine from the same person is analyzed by both wetting and immersion methods.

50 diapers with integrated urine accumulator and 50 devices with integrated urine strips manufactured by the company DiaSys Group pour Palma Group S.A. are used for the study. In these devices are incorporated urine strips comprising indicator reagents for glucose, ketones and PH. Analogous urine strips produced by DiaSys Group pour Palma Group S.A. are used to perform the analysis by traditional means.

The operating principle and studies of the diaper for the collection of biological samples are described in detail in the documents accompanying the patent application CH 00429/17.

The study is carried out as follows. Under the conditions of a clinical laboratory, urine from the same person is divided into two equal parts which are then studied simultaneously for three components: glucose, ketones and PH. The results obtained by this method of wetting fully correspond to those obtained by the usual method of immersion of the urine strips in a container containing the urine, which confirms the validity of the proposed method.

In the case of wetting, the color of indicator reagents changes after one minute.

The study of the validity of the data relating to the identification of leucocytes and protein by the moistening method is carried out under the same conditions, with urine accumulator diapers and urine strips from AllTest Biotech.

The urine from the same person is analyzed by the wetting and immersion methods. The validity of the results is confirmed by the microscopic study of the deposit.

The data obtained confirm the validity of the results obtained by the wetting method in 80% of the cases in comparison with the results obtained by the traditional method and by the microscopization of the deposit. The determination of the causes of error of the results shows that they are related to the structure of the tissue of the diaper accumulator which retains the insoluble elements in the urine, which demonstrates the effectiveness of the analysis of the urine by the wetting method for the identification of leukocytes and protein in the urine.

The causes of error of the results depends, in particular, on the compactness of the fibers of the urine accumulator.

1. A method for identifying deviations from a norm of one or more urine compounds and physico-chemical parameters, the method comprising:
A step (A) of separating a urine accumulator (200) from a diagnostic diaper (100),
A step (B) of impregnating a urine indicator strip (301), comprising one or more indicator reagents (307), by wetting via the urine accumulator (200), and
A reading step (C) of the result of the analysis,
wherein the diagnostic diaper (100) comprises an impermeable outer envelope (103) pierced with one or more orifices (104) with respect to a central area (102), the urine accumulator (200) being removably disposed outside the diaper (100) over the orifices (104), the urine accumulator (200) comprises a removable fastening element (201) and an absorption device (202), the absorption device (202) comprises a fastening interface (203) which holds it associated with the removable fastening element (201), the absorption device (202) being integral with the removable fastening element (201),
and that the method comprises the intermediate steps of separation (A1) of the removable fastening element (201), removal (A2) of the absorption device (202) integral with the removable fastening element (201), and separating (A3) the absorption device (202) from the attachment interface (203).

2. The method according to claim 1, wherein the separation step includes the detachment of a removable fastening element (201) enabling the removal of an absorption device (202) soaked in the urine to be analyzed.

3. The method according to claim 2, wherein the removable fastening element (301) is disposed in the central and an outer area of the diaper (100), thus allowing access to a sample of urine without having to remove the diaper (100).

4. The method according claim 1, wherein the urine indicator strip (301) is associated with, or arranged on, an analysis device (300), comprising a test area (304), an area having one or more colorimetric scales (306), an area (305) for disposing the urine accumulator (200), an area (308) for providing a moisture absorber, and an extrusion area (303) in relief.

5. The method according to claim 1, wherein step of impregnation of the urine indicator strip (301) by wetting via the urine accumulation element (200) is maintained for 10 to 120 seconds.

6. The method according to claim 1, wherein the step of reading the results comprises a time delay of 0 to 120 seconds.

7. The method according to claim 1, wherein the step of reading of the result includes a comparison of a color of the indicator reagents (307) with a corresponding color scale (306).

8. The method according to claim 1, wherein the step of reading of the result includes the recognition of a color of the indicator reagents (307) by an electronic device (400) equipped with an interface (401).

9. A diagnostic diaper (100), comprising a urine accumulator (200) disposed outside the diaper (100) and in a central position, and which is removably attached to the diaper (100) by a removable fastening element (201), wherein the diagnostic diaper (100) comprises an impermeable outer envelope (103) pierced with one or more orifices (104) opposite a central area (102), the urine accumulator (200) being removably disposed outside the diaper (100) over the at least one orifice (104), the urine accumulator (200) comprises a removable fastening element (201) and an absorption device (202), the absorption device (202) comprises a fastening interlace (203) which holds it associated with the removable fastening element (201), the absorption device (202) being integral with the removable fastening element (201).

10. A kit comprising the diaper as described in claim 9, and at least one urine strip (301).

11. The kit according to claim 10, further comprising an explanatory note or a reference to a downloadable program on an electronic device (400), or a combination of both.
